(19) United States
(12) Patent Application Publication
(10) Pub. No.: US 2004/0092739 A1
(43) Pub. Date: May 13, 2004

(54) Process for synthesizing antifolates

(76) Inventors: Zejun Xiao, San Antonio, TX (US); Harry Kochat, San Antonio, TX (US)

Correspondence Address: Thomas J. Dodd, BioNumerik Pharmaceuticals, Inc., Suite 1250, 8122 Datapoint Drive, San Antonio, TX, 78229, US

(21) Appl. No.: 10/627,485

(22) Filed: Jul. 25, 2003

Related U.S. Application Data
(60) Provisional application No. 60/425,826, filed on Nov. 13, 2002.

(51) Int. Cl. C07D 487/02; C07D 239/70; C07D 475/02

(52) U.S. Cl. 544/184; 544/236; 544/256; 544/257; 544/280; 544/283

(57) ABSTRACT

This invention relates to a process for synthesizing certain folic acid analogues, which are useful in treating cancer, inflammatory diseases, autoimmune diseases, and are commonly referred to as antifolates. The process employs improved steps for annulation, derivatization and addition reactions to produce the described antifolates from commonly available starting materials.

### CROSS REFERENCE TO RELATED APPLICATIONS

This application claims the benefit of U.S. Provisional Application Serial No. 60/425,826, filed Nov. 13, 2002, which is incorporated herein by reference.

### FIELD OF THE INVENTION

This invention relates to a process for synthesizing antifolates, and will have application to a process for synthesizing compounds and intermediates for making—methylene-10-deaza aminopterin (MDAM) and the 5,8-dideaza analogues thereof.

### BACKGROUND OF THE INVENTION

Antifolates comprise a well-known class of compounds that have exhibited beneficial medicinal properties in several therapeutic areas. Antifolates have been used for many years as treatments for various cancers, infectious diseases, immunosuppression, inflammatory diseases and others.

Antifolates are so named because of their mode of action, by interfering with the folic acid metabolic pathway. The most well known antifolate, methotrexate (MTX), inhibits dihydrofolate reductase (DHFR), thus preventing the reduction of folic acid to its dihydro and tetrahydro forms. Other antifolates, such as aminopterin (AMT), MDAM and others also act by inhibiting DHFR, while still others, such as MTX polyglutamates, act at different areas of the folic acid pathway, most notably the thymidylate synthetase (TS) inhibitors, Glucineamide Ribonucleotide (GAR) and Aminoimidazole Carboxamide Ribonucleotide (AICAR) inhibition.

Most antifolates used in oncology are similar in chemical structure to the naturally occurring vitamin, folic acid, the structure of which is shown below, along with a few other widely known antifolate structures.

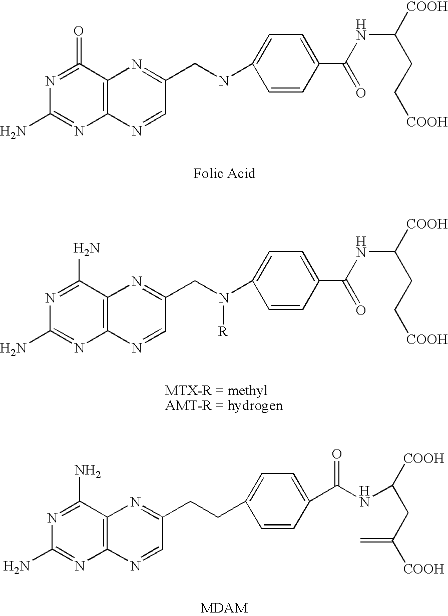

U.S. Pat. No. 5,912,251 discloses an antifolate compound, hereinafter referred to as 5,8-dideaza MDAM that is similar in structure to MDAM. The structure of 5,8-dideaza MDAM (hereinafter referred to as gamma methylene glutamate 5,8,10-trideaza aminopterin or TRIDAM) is shown below as Formula A.

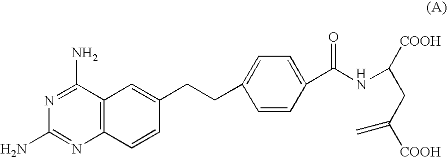

A major mode of action of TRIDAM is TS inhibition in addition to some degree of DHFR inhibition. It has been postulated in the '251 patent that TRIDAM may find application not only in oncology, but also in other medical areas that antifolates have found success. Asthma, rheumatoid arthritis, psoriasis, and other inflammatory diseases are potential targets for TRIDAM.

The previous process for synthesizing TRIDAM and analogues thereof, as disclosed in the '251 patent, is inefficient and not commercially viable due to low overall yields and the impractical application or costs of various reagents and procedures used; the overall yield from the process is less than one percent of the starting materials. The '251 process is depicted below as Scheme A.

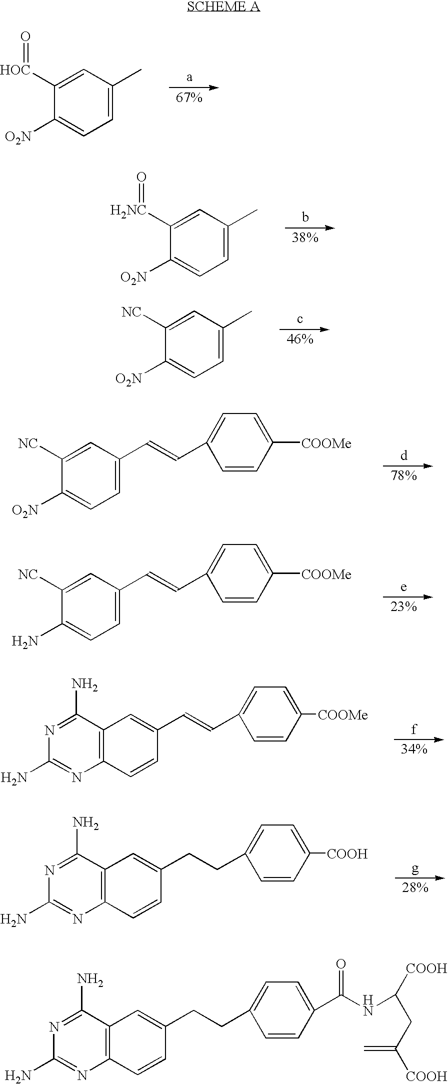

Overall Yield: 0.20%

a—i-BuOC(═O)Cl, Et₃N, NH₃, DCM

b—POCl₃, DMF

c—Methyl 4-formylbenzoate, NaOMe, MeOH

d—Na₂S₂O₄, DMF

e—Guanidine

f—1. H₂/Pd—C, DMF

2. 0.1N NaOH

g—1. Et₃N/i-BuOC(═O)Cl/DMF

2. diethyl L-glutamate hydrochloride

3. 0.1N NaOH

5 M. G. Nair, U.S. Pat. No. 5,912,251 (1999)

Another reported synthetic process for making a close analogue of TRIDAM (identical in all respects except for the amino acid residue) was disclosed by Harris, et al. in 1990 and is shown below as Scheme B.

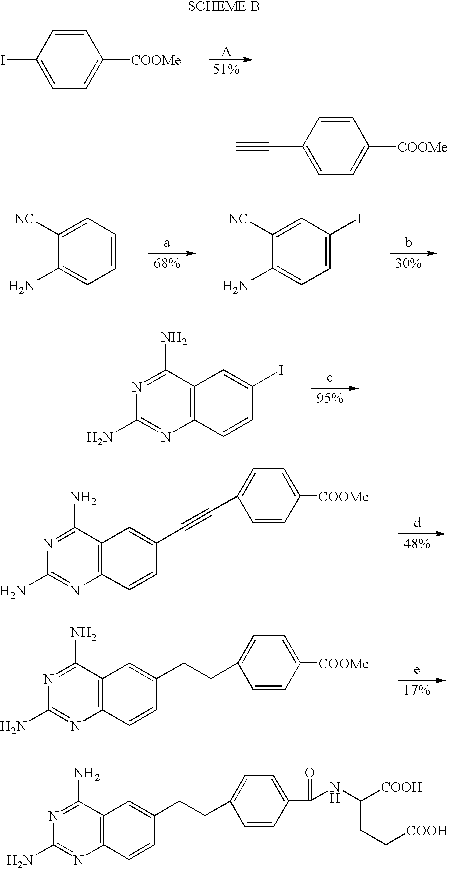

Overall Yield: 0.8%

A—1. Me₃SiC≡CH/Pd(OAc)₂/P(Ar)₃/CuI/Et₃N

2. K₂CO₃ (cat.)/MeOH

a—ICl/AcOH

b—NH₂C(═O—NH)Cl.HCl/diglyme

c—Pd(OAc)₂/P(Ar)₃/CuI/Et₃N/DMF

d—H₂/Pd-C/AcOH/DMF

e—1. 1.0M KOH/MeOCH₂CH₂OH

2. Et₃N/i-BuOC(═O)Cl/DMF, then diethyl L-glutamate hydrochloride

3. 1.0M KOH/MeOCH₂CH₂OH

N. V. Harris et al, Synlett., 577 (October 1990)

### SUMMARY OF THE INVENTION

The process of this invention provides for an efficient and economical process for synthesizing TRIDAM and intermediates thereof, together with certain analogues, derivatives and/or congeners thereof.

The critical intermediate synthesized according to the process of this invention is the analogue of pteroic acid, shown below as Formula I.

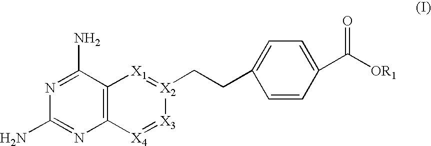

where R₁ is hydrogen, lower alkyl, or any oxygen protecting group, and X₁-X₄ are each individually carbon or nitrogen.

Once intermediate I has been synthesized, known methods may be utilized to couple an amino acid residue to the molecule to form the desired antifolate compound. Preferred amino acids are glutamic acid, aspartic acid and their derivatives, most preferably the naturally occurring L-enantiomer, but other amino acids may also be employed.

The process of this invention reduces the steps required in the prior art to synthesize the formula I compounds from commercially available starting materials, using commercially available reagents. Overall yields are also increased. The process includes the initial derivatization and then annulation of the fused heterocyclic portion of the formula I compounds. A leaving group is added to the heterocycle, followed by addition of the p-benzoic acid alkylene linker, followed by coupling of the amino acid side chain. The process is described in detail in the foregoing schemes and examples.

An object of this invention is to provide for an efficient and economical process for synthesizing antifolate compounds.

### DESCRIPTION OF THE PREFERRED EMBODIMENT

The preferred embodiment herein described is not intended to be exhaustive or to limit the invention to the precise form disclosed. It is chosen and described to explain the principles of the invention, and its application and practical use to enable others skilled in the art to follow its teachings.

The process of this invention provides for the synthesis of compounds having the formula Ia below:

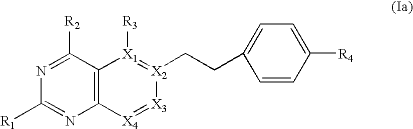

wherein R₁ and R₂ are each individually amino or N-alkyl substituted amino; hydroxy; alkoxy; keto; lower alkyl; or a nitrogen or oxygen protecting group;

R₃ is hydrogen; hydroxy; alkoxy; trifluoromethyl alkoxy; halo; sulfhydryl or alkylthio;

R₄ is hydroxy; alkoxy; or —C(O)—X;

X is hydroxy; alkoxy; an amino acid residue; and

X₁-X₄ are each individually carbon or nitrogen.

The formula I compounds are commonly referred to as antifolates, because of their inhibitory effects on the folic acid nutritional pathways. For purposes of identification, the formula I antifolates are possessed of three linked moieties: (i) a 2,4 (5) di(tri)substituted heterocyclic moiety; (ii) a p-benzoic acid alkylene moiety; and (iii) an amino acid residue. The moieties as described above are shown below as Formula Ib:

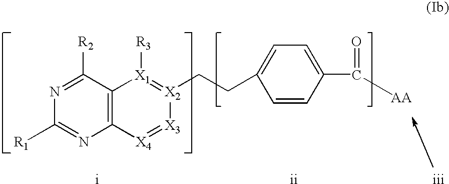

where AA is the amino acid residue.

The following scheme generally illustrates the process of this invention.

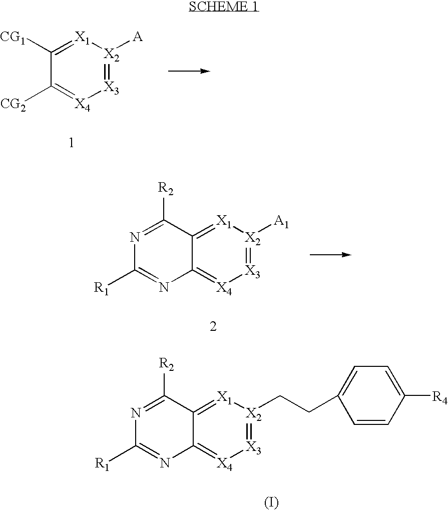

As shown in Scheme 1, the process to synthesize the critical intermediate end product involves two general steps, each step preferably including multiple steps to achieve the desired result. In the scheme, CG₁ and CG₂ are moieties capable of reacting with an annulation agent to form the desired fused ring heterocycle, A₁ is a leaving group, and the R and X variables have the same meaning as in formula I.

The starting material is first annulated and if necessary, derivatized to add leaving group A₁, to form intermediate compound 2. Preferred annulation groups include guanidine or a derivatized guanidine, or other known reagents. Derivatization, if necessary, is employed using conventional techniques to add the leaving group A₁, which is preferably a halogen group, but may be any suitable leaving group.

Intermediate 1 is then converted to the desired end product in one or two steps through a modified Wittig reaction. If the desired R₄ value is an amino acid, the amino acid residue may be coupled to the p-benzoic acid moiety by any known process, such as the processes described above. It should be noted that, if desired, reactable moieties may be protected by conventional means prior to any of the steps of the inventive process.

The following examples are illustrative of the process of this invention.

### EXAMPLE 1

### 5-Methyl-2-nitrobenzamide

To a solution of 5-methyl-2-nitrobenzoic acid (50.0 g, 0.276 mol) in dichloromethane (1380 mL) and triethyl amine (50.24 mL, 1.3 equiv) was added isobutyl chloroformate (43.0 mL, 1.2 equiv) over syringe at −10° C. The ice bath was removed and the reaction solution in dark red color was stirred at room temperature for 2 hours (TLC monitored). Ammonia was bubbled in the solution for 2 hours until a strong basic solution resulted (pH 10). Brown solid was formed and the resulting suspension was stirred for 18 hours at room temperature (TLC monitored the reaction). The reaction was quenched by addition of 1000 mL of saturated sodium bicarbonate aqueous solution. The mixture was extracted with ethyl acetate (1500 mL, 3×1000 mL). Vigorous shaking was performed during extraction. The combined organic layers were dried with sodium sulfate and concentrated to give a dark brown solid, which was recrystallized from ethyl acetate at 0° C. for 14 hours to give 29.8 g (60%) of brown solid. The mother solution was concentrated and kept at 0° C. to give the second crop of product (7.5 g, 15%, combined yield 75%). ¹H NMR (Acetone-d₆): δ 2.47 ppm (s, 3H, CH₃), 6.95 (s, br, NH₂), 7.45 (d, 2H, aromatic), 7.90 (d, 2H, aromatic).

### EXAMPLE 2

### 5-Methyl-2-nitrobenzonitrile

To a solution of 5-methyl-2-nitrobenzamide from Example 1 (29.8 g, 0.165 mol) in 329.6 mL of N,N-dimethyl formamide was added phosphorous oxychloride (16.96 mL, 1.1 equiv) through syringe over 20 min. at −10° C. The resulting mixture was stirred at 25° C. for 40 minutes, then heated and stirred at 100° C. for 15 minutes. The reaction mixture was poured into ice (750 g) and ammonia (75 mL) was added to the resulting suspension until pH of aqueous solution reached between 9-10. The aqueous layer was extracted with ethyl acetate (1000 mL, 2×600 mL). The combined organic layers were dried over sodium sulfate and concentrated to give a yellow solid (25.7 g, 96%), which was used directly to next step without further purification. ¹HNMR spectrum confirmed the presence of substantially pure title compound. ¹H NMR (CDCl₃): δ 2.54 ppm (s, 3H, CH₃), 7.59 (d, J=8.4 Hz, aromatic), 7.71 (s, aromatic), 8.24 (d, J=8.4 Hz, aromatic).

### EXAMPLE 3

### 2-Amino-5-methylbenzonitrile

To a solution of 5-methyl-2-nitrobenzonitrile from Example 2, (25.7 g, 0.158 mol) in 643 mL of acetonitrile was added sodium dithionate (128.5 g, 0.739 mol), followed by addition of 600 mL of deionized water at 0° C. The reaction mixture was stirred for 30 minutes at 25° C. The aqueous layer was extracted with ethyl acetate three times, and the combined organic layers were dried over sodium sulfate and evaporated under vacuum to afford a crude yellow solid, which was dried under high vacuum for 24 hours to give 16.4 g of substantially pure title compound (78.4%). ¹H NMR (CDCl₃): δ 2.23 ppm (s, 3H, CH₃), 6.65 (d, J=8.4 Hz, aromatic), 7.18 (s, aromatic), 7.14 (d, J=8.4 Hz, aromatic).

### EXAMPLE 4

### 2,4-Diamino-6-methylquinazoline

A mixture of 2-amino-5-methylbenzonitrile from Example 3 (47.0 g, 0.356 mol) and cyanoguanidine (37.4 g, 1.25 equiv) in 355 mL of 1N hydrochloric acid aqueous solution was heated at reflux for 1.5 hours. 828 mL of deionized water and 355 mL of 1N hydrochloric acid were added to the reaction mixture. The mixture was filtered while hot. The filtrate was neutralized with 473 mL of 2N sodium hydroxide aqueous solution and the resulting yellow precipitate was filtered. 573 ML of deionized water was added to the yellow solid, followed by addition of 95 mL of formic acid. The resultant suspension was stirred for 2 hours and the white precipitate was filtered. 1.6 L of deionized water was added and 154 mL of ammonium hydroxide was added to the white solid. The suspension was stirred for 1 hour. The pale yellow solid was filtered and dried under high vacuum to give 22.0 g of substantially pure title compound. ¹H NMR (Acetone-d₆): δ 2.35 ppm (s, 3H, CH₃), 5.42 (s, br, NH₂), 6.63 (s, br, NH₂), 7.20 (d, aromatic), 7.38 (dd, aromatic), 7.77 (s, aromatic).

### EXAMPLE 5

### 2,4-Dibenzamido-6-methylquinazoline

To a suspension of 2,4-diamino-6-methylquinazoline from Example 4 (34.0 g, 0.195 mol) and anhydrous triethyl amine (136 mL, 5 equiv) in 1 L of 1,4-dioxane was added benzoyl chloride (50.6 mL, 2.5 equiv) at reflux for 30 minutes. The resultant mixture was stirred for 30 minutes at reflux, and solid was filtered and washed with hot 1,4-dioxane. The filtrate was concentrated and the crude solid was recrystallized from ethanol to give 61.0 g of the title product (82%). ¹H NMR (CDCl₃): δ 2.56 ppm (s, 3H, CH₃), 7.53 (m, aromatic), 8.08 (d, 2H, aromatic), 8.60 (d, 3H, aromatic).

### EXAMPLE 6

### 2,4-Dibenzamido-6-bromomethylquinazoline

A refluxing mixture of 2,4-dibenzamido-6-methylquinazoline from Example 5 (19.1 g, 0.05 mol), 1,3-dibromo-5,5-dimethyl-imidazolidine-2,4-dione (8.50 g 0.60 equiv) and 1.40 g of benzoyl peroxide in 1 L of carbon tetrachloride was irradiated with a high intensity lamp (600 W, 120V). The reaction mixture was kept at this condition for 1 hour. The mixture was allowed to cool to 25° C. and saturated sodium bicarbonate aqueous solution was added and stirred for 1 hour. Solid was filtered, washed with ether, and dried under high vacuum to give 24.6 g of crude end product (82% from proton NMR), which was used for next step without further purification. ¹H NMR (CDCl₃): δ 4.68 ppm (s, 2H, CH₂), 7.57 (m, aromatic), 7.82 (dd, 1H, aromatic), 8.08 (d, 2H, aromatic), 8.56 (d, 2H, aromatic). 8.73 (s, 1H, aromatic). HRMS calcd for C₂₃H₁₈N₄O₂ 382.14, found 383.14072 (protonated).

### EXAMPLE 7

### 2,4-Dibenzamido-6-(p-methoxycarbonyl) phenylvinylquinazoline

A mixture of 2,4-dibenzamido-6-bromomethylquinazoline from Example 6 (19.68 g, 42.66 mmol) and triphenylphosphine (12.31 g, 1.1 e) in 427 mL of tetrahydrofuran was heated at reflux for 2 hours. The reaction mixture was allowed to cool to 25° C. and the precipitate was filtered. To this white solid was added 3.81 g of methyl 4-formylbenzoate and 220 mL of tetrahydrofuran (THF). The resultant mixture was stirred at −10° C. for 20 minutes and potassium t-butoxide (1M in THF, 44.22 mL) was added. The reaction mixture was stirred at 25° C. for 24 hours, and saturated aqueous sodium bicarbonate was added. The aqueous layer was extracted three times with ethyl acetate. The combined organic layers were washed with brine, dried over sodium sulfate, and evaporated to give a crude yellow oil, which was treated with ethyl acetate to yield 12 g of the title product. ¹H NMR (CDCl₃): δ 3.94 ppm (s, 3H, OCH₃), 7.30 (d, 1H, olefin), 7.39 (s, 1H, olefin), 7.57 (m, aromatic), 7.67 (d, 2H, aromatic), 8.08 (m, aromatic), 8.58 (d, 2H, aromatic). 8.80 (s, 1H, aromatic).

### EXAMPLE 8

### 2,4-Dibenzamido-6-(p-methoxycarbonyl) phenethylquinazoline

A mixture of the olefin from Example 7 (7.0 g, 13.2 mmol) and 10% palladium on carbon (700 mg, 10%) in 400 mL of DMF was hydrogenated for 20 hours at a hydrogen pressure of 20 psi. The catalyst was removed by filtration over celite and the filtrate was evaporated to give 6.5 g of pure title product. ¹H NMR (CDCl₃): δ 3.05 ppm (m, 4H, CH₂CH₂), 3.88 (s, 3H, OCH₃), 7.53 (m, aromatic), 8.02 (m, 4H, aromatic), 8.52 (d, 2H, aromatic).

### EXAMPLE 9

### 4-Amino-4-deoxy-5,8,10-trideaza Pteroic Acid

A mixture of the hydrogenation product from Example 8 (6.5 g, 12.3 mmol), 183 mL of 1 N KOH, and 123 mL of acetonitrile was heated at reflux for 42 hours. The reaction solution was neutralized with acetic acid at 25° C. The resulting white precipitate was filtered, washed with a solution of acetonitrile and water, and dried to give 3.7 g of the desired title product. ¹H NMR (DMSO, d₆): δ 2.90 ppm (m, 4H, CH₂CH₂), 6.08 (s, br, NH₂), 7.03 (d, 2H, aromatic), 7.2 (m, 5H, aromatic), 7.76 (d, 2H, aromatic), 7.80 (s, 1H, aromatic). HRMS calcd for C₂₃H₁₈N₄O₂H⁺ 309.134602, found 309.13477 (protonated).

### EXAMPLE 10

### Diethyl 4′-methylene-N-[2,4-diamino-quinazolin-6-ethyl(4-benzoyl)] glutamate (Diethyl Ester of TRIDAM)

To a suspension of 3.4 g of 4-amino-4-deoxy-5,8,10-trideaza pteroic acid from Example 9 in 80 mL of DMF was added 3.6 g of L-diethyl-4-methylene glutamate hydrochloride, 0.34 g of 1-hydroxy benzotriazole, and 4.23 g of 1-[(3-dimethylamino)propyl]3-ethyl carbodiimide hydrochloride. The mixture was stirred for 30 minutes at 25° C., and 3.10 mL of anhydrous triethylamine was added through syringe. The reaction mixture was stirred at 25° C. for 18 hours. HPLC monitored the reaction until no starting material was observed. The reaction mixture was poured into 300 g of ice. The white precipitate was filtered and dried to afford 5.5 g of the title product (99%). ¹H NMR (DMSO, d₆): δ 1.12 (m, 6H, CH₃), 2.64 (m, 1H), 2.88 (m, 5H), 4.06 (m, 4H, CH₂), 4.57 (m, 1H), 5.63 (s, 1H, olefin), 5.83 (s, br, NH₂), 6.05 (s, 1H, olefin), 7.04 (d, 1H, aromatic), 7.14 (s, br, NH₂), 7.24 (d, 2H, aromatic), 7.32 (d, 1H, aromatic), 7.68 (d, 2H, aromatic), 8.53 (d, 1H, aromatic). 7.79 (s, 1H, aromatic). HRMS calcd for C₂₇H₃₁N₅O₅Na⁺ 528.221738, found 528.22225.

### EXAMPLE 11

### 4′-Methylene-N-[2,4-diamino-quinazolin-6-ethyl(4-benzoyl)] glutamic Acid (TRIDAM)

A mixture of diethyl-4′-methylene-5,8,10-trideazaminopterin from Example 10 (5.5 g, 11 mmol), 544 mL of 1 N NaOH, and 220 mL of acetonitrile was stirred at 25° C. for 16 hours. The reaction solution was neutralized with acetic acid at 25° C. The resulting white precipitate was filtered, washed with a solution of acetonitrile and water, and dried to give 4.2 g (85%) of the title product. ¹H NMR (DMSO, d₆): δ 2.58 (m, 1H), 2.84 (m, 5H), 4.44 (m, 1H), 5.5 (s, 1H, olefin), 5.95 (s, 1H, olefin), 6.88 (s, 2H, NH₂), 7.20 (dd, 3H, aromatic), 7.41 (d, 1H, aromatic), 7.75 (d, 2H, aromatic), 7.95 (s, 1H, aromatic), 9.03 (s, br, COOH).

The above description is illustrative of the process of this invention, is not limitative thereof, and may be modified within the scope of the following claims.

What is claimed is:

1. A process for synthesizing compounds having the formula: 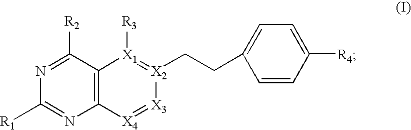
wherein R₁ and R₂ are each individually amino or N-alkyl substituted amino; hydroxy; alkoxy; keto; lower alkyl; or a nitrogen or oxygen protecting group;
R₃ is hydrogen; hydroxy; alkoxy; trifluoromethyl alkoxy; halo; sulfhydryl or alkylthio;
R₄ is hydroxy; alkoxy; or —C(O)—X;
X is hydroxy; alkoxy; or an amino acid residue; and
X₁, X₂, X₃ and X₄ are each individually carbon or nitrogen;
said process comprising the steps of:
a) providing a starting compound of the formula: 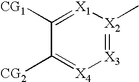
where CG₁ and CG₂ are moieties amenable to an annulation reaction;
b) annulating the starting compound to form a 1N, 3N two-ring fused heterocycle;
c) derivatizing the two-ring fused heterocycle at the 6-position to form an intermediate: 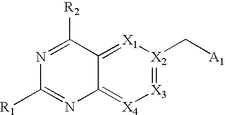
where A₁ is a leaving group;
d) adding a derivative of p-benzoic acid to the intermediate to form the formula I compound.
